(19) United States
(12) Patent Application Publication
(10) Pub. No.: US 2023/0381807 A1
(43) Pub. Date: Nov. 30, 2023

(54) APPARATUS AND METHOD FOR MANUFACTURING COSMETIC OR MEDICAL FUNCTIONAL PATCH

(71) Applicants: ANY TAPE Co., Ltd., Hwaseong-si (KR); Anymedi & Health Care Co., Ltd., Hwaseong-si (KR); Duogreen Inc., Northvale, NJ (US)

(72) Inventor: Sungho PARK, Hwaseong-si (KR)

(21) Appl. No.: 18/202,886

(22) Filed: May 26, 2023

(30) Foreign Application Priority Data
May 31, 2022 (KR) 10-2022-0066931

(51) Int. Cl. B05C 5/02

(52) U.S. Cl. CPC B05C 5/025; B05C 5/0225

(57) ABSTRACT

The present disclosure relates to an apparatus and method for manufacturing a functional patch for cosmetic or medical use, and more particularly, to an apparatus and method for manufacturing a functional patch for cosmetic or medical use by applying a functional material to a base patch and then drying it. The functional patch manufacturing apparatus according to the present disclosure includes a dispensing section for applying a functional material to a multi-layer patch film in which the base patch is disposed, so that the functional material can be applied accurately to the base patch, which minimizes a defect rate of the product. Further, the functional patch manufacturing apparatus includes a drying section and a recovery sections addition to the dispensing section, so that it is possible to maximize production capacity per unit time.

### TECHNICAL FIELD

The present application claims the benefit of priority to Korea Patent Application No. 10-2022-0066931, titled “Cosmetic or medical functional patch manufacturing equipment and manufacturing method therefor, filed with the Korea National Intellectual Property Administration on May 31, 2022, the entire disclosure of which is incorporated herein by reference.

The present disclosure relates to an apparatus and method for manufacturing a cosmetic or medical functional patch, and more particularly, to an apparatus and method for manufacturing a cosmetic or medical patch by applying a functional material to a base patch and then drying the same.

### BACKGROUND

Recently, people's interest in beauty and the like has increased and various functional products have been released according to the growth of related industries. Among them, a hydro-colloidal patch is widely used as a moisture-retentive dressing bandage because it is thin, has excellent adhesiveness, and can prevent drying of exudate and other fluids at a wound site.

However, such a conventional hydro-colloidal patch only has a function of absorbing exudate in contact with the wound, and does not contain active ingredients that can directly treat the wound. Further, even when used on a skin for cosmetic or medical purposes, conventional hydro-colloidal patch only performs the function of maintaining moisture in the skin, and does not contain any active ingredients for skin beauty or treatment.

In addition, a conventional mask pack for skin care or treatment does not have a separate adhesive force, so the moisture of the mask pack evaporates over time after the mask pack is attached, and the skin adhesive force is rapidly reduced. Therefore, it is difficult to attach the mask pack to a local area for a long time.

A patch containing a functional material may be considered to solve the above problem. In order to manufacture the patch containing such a functional material, it is necessary to be able to accurately apply the functional material to the patch, and also to be able to dry and recover the applied functional material at an appropriate temperature within a short period of time.

### SUMMARY

In view of the above, the present disclosure provides a functional patch manufacturing apparatus and method for manufacturing a functional patch that minimizes the defect rate of the product by accurately applying a functional material to a base patch and maximizes the production capacity per unit time.

A functional patch manufacturing apparatus according to one embodiment of the present disclosure includes a dispensing section, a drying section, and a recovery section and manufactures a functional patch by processing a plurality of base patches disposed in a multi-layer patch film. The multi-layer patch film includes: a base film having a width and extending in a longitudinal direction; a guide film disposed on the base film, the guide film having at least one guide hole smaller than the base patch, having a width less than or equal to that of the base film, and having a length shorter than that of the base film; and a cover film formed and disposed to cover the base film and the guide film. The base patch is attached to the guide film and the base film at a position corresponding to the guide hole between the guide film and the base film, so that at least a part of the base patch is exposed to the outside of the guide film. The base film, the base patch, the guide film and the cover film are sequentially stacked and disposed. The dispensing section includes: a main roller on which the multi-layer patch film is initially wound, the wound multi-layer patch film being unwound from the main roller during operation; a separation roller for separating the cover film from the multi-layer patch film unwound from the main roller; a cover film collection roller for collecting the cover film separated from the separation roller; a dispenser for applying a functional material to a portion of the base patch exposed through the guide hole in the multi-layer patch film separated by the separation roller from above; a functional material supply device for supplying the functional material to the dispenser; a controller for controlling the application operation of the dispenser and the supply operation of the functional material supply device. The drying section includes a heat supply unit for supplying heat to the functional material applied to the base patch. The base patch is converted into a finished patch in which the functional material is dried by the heat supplied from the heat supply unit to be discharged. The recovery section includes a recovery roller for recovering the multi-layer patch film in which the finished patch is disposed in a state in which the cover film is removed.

The base patch may include a base material film and an adhesive layer bonded to the base material film, and the functional material may be applied to the adhesive layer.

The base material film may be a thermoplastic polyurethane resin material, and the adhesive layer may be a hydrocolloid layer.

The functional material may contain 85 to 97% by weight of hyaluronic acid.

In the guide film, n number of guide holes may be formed in a row in a width direction of the base film, and the dispenser may include n number of nozzles disposed to correspond to the guide holes of the row, and the n number of nozzles may simultaneously apply the functional material.

In the guide film, n number of guide holes may be formed in a row in a width direction of the base film, and m number of guide holes may be formed in a column in a longitudinal direction of the base film.

The controller may control the dispenser to sequentially repeat an application step in which the dispenser applies the functional material m times at a first time interval and a stopping step in which the application of the dispenser is stopped for a second time after the application step.

The functional patch manufacturing apparatus may further include: a thickness sensor for measuring the thickness of each part of the multi-layer patch film in real time, wherein the controller may analyze a thickness value measured by the thickness sensor, and control the dispenser so that the dispenser applies the functional material when it is determined that the guide hole is at a position corresponding to the dispenser.

The heat supply unit may be a hot air supply unit for blowing hot air to the functional material applied to the base patch.

The heat supply unit may be a near-infrared heater for supplying near-infrared radiant heat to the functional material applied to the base patch.

The functional patch manufacturing apparatus according to the present disclosure includes the dispensing section for applying the functional material to the multi-layer patch film in which the base patch is disposed, so that the functional material can accurately be applied to the base patch to minimize the defective rate of the product, and further includes the drying section and the recovery section in addition to the dispensing section, so that the production capacity per unit time can be maximized.

### BRIEF DESCRIPTION OF THE DRAWINGS

FIG. 1 is a perspective view showing a part of a multi-layer patch film to be processed by a functional patch manufacturing apparatus according to one embodiment of the present disclosure by cutting it.

FIG. 2 is a front view showing the functional patch manufacturing apparatus according to one embodiment of the present disclosure.

FIG. 3 is an enlarged front view showing a dispensing section in FIG. 2.

FIG. 4 is an enlarged front view showing a drying section in FIG. 2.

FIG. 5 is an enlarged front view showing a recovery section in FIG. 2.

FIG. 6 is a perspective view showing the operation of the dispensing section according to one embodiment of the present disclosure.

FIG. 7 is a perspective view showing the operation of a dispensing section according to another embodiment of the present disclosure.

FIG. 8 shows a drying section including a near-infrared heater according to still another embodiment of the present disclosure.

### DETAILED DESCRIPTION

The present disclosure may be variously modified and have various embodiments, and specific embodiments will be exemplified and described in detail in the detailed description. However, it should be understood that this is not intended to limit the present disclosure to the specific embodiments, but includes all modifications, equivalents, and substitutes included in the idea and scope of the present disclosure. Terms used in the present disclosure are only used to describe the specific embodiments, and are not intended to limit the present disclosure. Singular expressions include plural expressions unless the context clearly indicates otherwise. In the present disclosure, it should be understood that terms such as ‘comprise’, ‘include’, or ‘have’ are intended to designate that there is a feature, number, step, operation, component, part, or combination thereof described in the present specification, but they do not preclude the possibility of the presence or addition of one or more other features, numbers, steps, operations, components, parts, or combinations thereof. Hereinafter, preferred embodiments of the present disclosure will be described in detail with reference to the accompanying drawings. In this case, it should be noted that in the accompanying drawings, the same components are indicated by the same reference numerals as much as possible. In addition, detailed descriptions of well-known functions and configurations that may obscure the gist of the present disclosure will be omitted. For the same reason, in the accompanying drawings, some components are exaggerated, omitted, or schematically illustrated.

Hereinafter, a functional patch manufacturing apparatus according to one embodiment of the present disclosure will be described in detail with reference to the accompanying drawings.

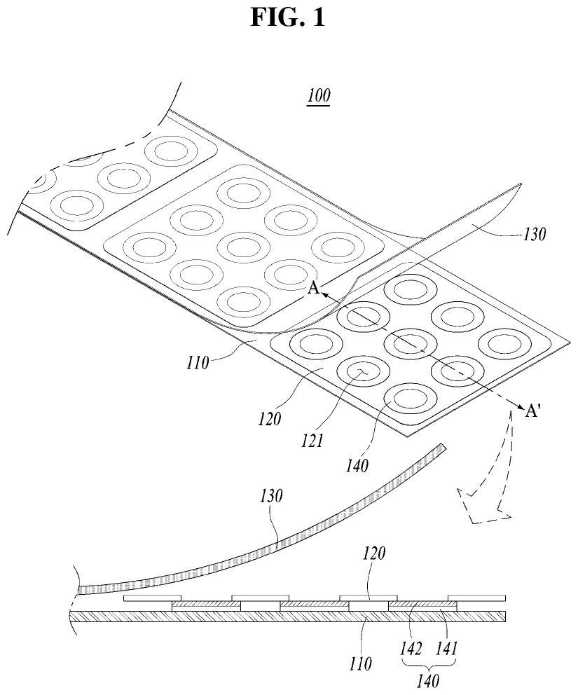

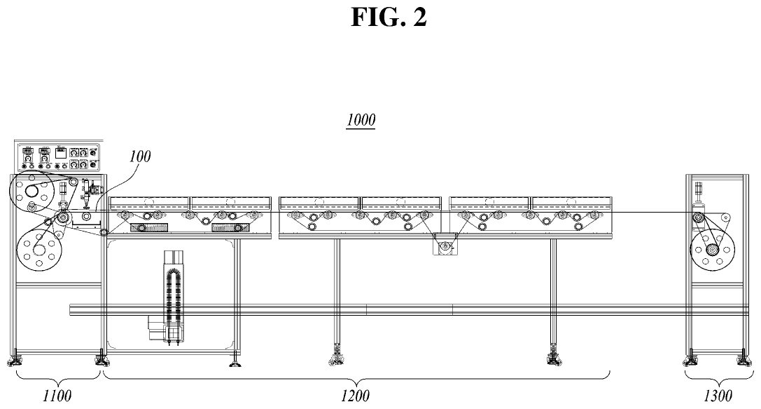

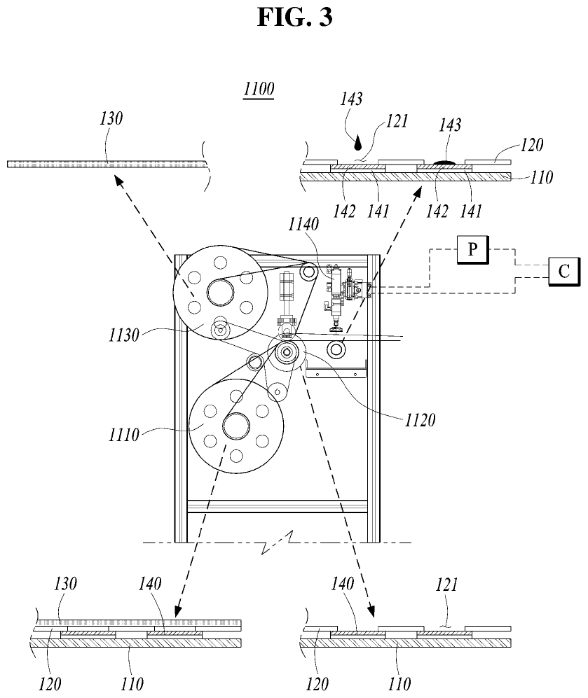

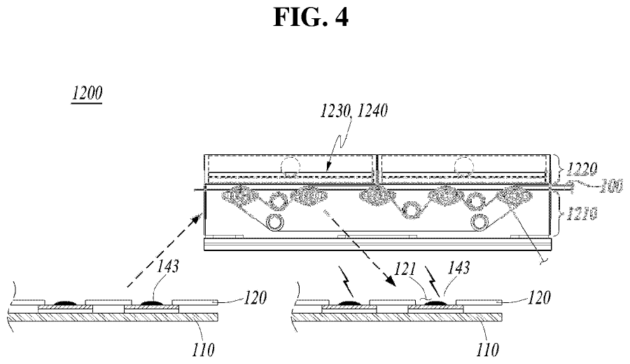

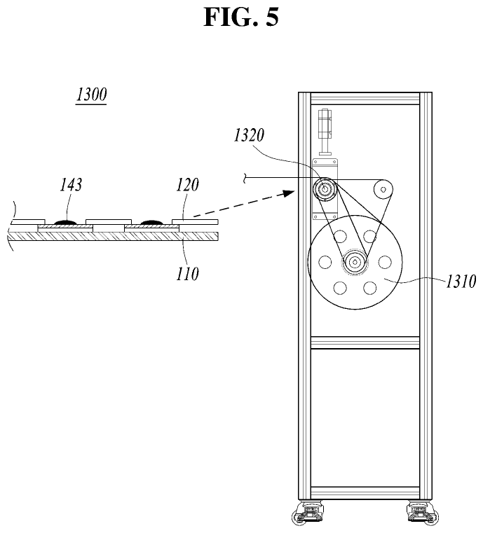

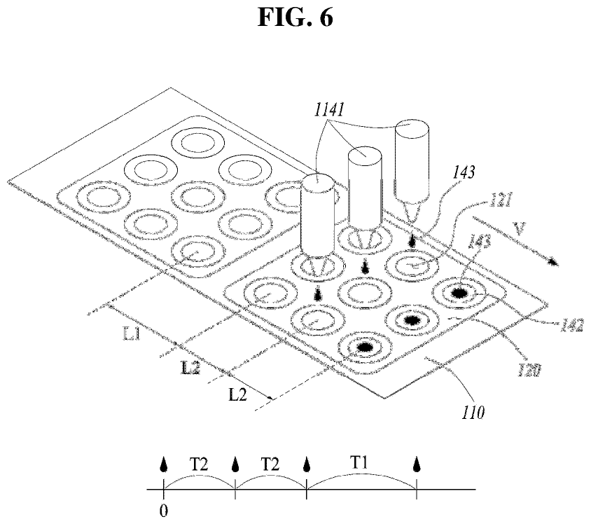

FIG. 1 is a perspective view showing a part of a multi-layer patch film to be processed by a functional patch manufacturing apparatus according to one embodiment of the present disclosure by cutting it, FIG. 2 is a front view showing the functional patch manufacturing apparatus according to one embodiment of the present disclosure, FIG. 3 is an enlarged front view showing a dispensing section in FIG. 2, FIG. 4 is an enlarged front view showing a drying section in FIG. 2, FIG. 5 is an enlarged front view showing a recovery section in FIG. 2, and FIG. 6 is a perspective view showing the operation of the dispensing section according to one embodiment of the present disclosure.

Hereinafter, with reference to FIG. 1, a multi-layer patch film 100, which is a processing target of a functional patch manufacturing apparatus 1000 according to one embodiment of the present disclosure, will be described in detail. Functional patch manufacturing apparatus 1000 according to one embodiment of the present disclosure is an apparatus for manufacturing a functional patch with functionality by processing the multi-layer patch film 100.

The multi-layer patch film 100 includes a base film 110, a guide film 120, a cover film 130, and a base patch 140. The base film 110 has a width and extends in a longitudinal direction. The base film 110 may be shaped like a long tape having a width.

The guide film 120 has a width smaller than that of the base film 110 and a length shorter than that of the base film 110. The guide film 120 is disposed on the base patch 140. At least one open guide hole 121 is formed in the guide film 120.

The guide hole 121 is formed to have a size smaller than the size of the base patch 140. The base patch 140 is attached to each guide hole 121. The number of the base patches 140 corresponds to the number of the guide holes 121 and the base patch 140 is disposed at a position corresponding to each guide hole 121. The base patch 140 is disposed at a position corresponding to the guide hole 121 between the guide film 120 and the base film 110. Accordingly, the base patch 140 is attached to both the guide film 120 and the base film 110, and at least a portion of the base patch 140 is exposed to the outside of the guide film 120 through the guide hole 121.

In the guide film 120, n number of guide holes 121 may be formed along a width direction of the base film 110 so that the n number of guide holes 121 form a row. In this case, n means a natural number greater than 1. In addition, m number of guide holes 121 may be formed along a length direction of the base film 110 so that the m number of guide holes 121 form a column. In this case, m means a natural number greater than 1, and m may be equal to n. Accordingly, the guide holes 121 may be formed in n rows and m columns in the guide film 120. In FIG. 1, n and m are each 3, but the present disclosure is not limited thereto.

The cover film 130 is formed and disposed to cover the base film 110 and the guide film 120. The cover film 130 may cover the base film 110 and the guide film 120 to protect the base film 110 and the guide film 120 from the outside before processing. Like the base film 110, the cover film 130 may be formed to have a width and to extend in the longitudinal direction. In some cases, the cover film 130 may be formed to have the same size as the base film 110 or may be formed to have a minimum size to cover only the base film 110.

As a result, the base patch 140 is disposed between the base film 110 and the guide film 120, and in this state, the base film 110, the base patch 140, the guide film 120, and the cover film 130 may be sequentially stacked and disposed.

The base patch 140 may include a base material film 141 and an adhesive layer 142, and a functional material 143 may be applied to the adhesive layer 142. The adhesive layer 142 is bonded to the base material film 141. The base material film 141 may include a thermoplastic polyurethane resin, and in this case, it may be preferable in terms of elasticity.

The adhesive layer 142 may be a hydrocolloid layer and may include a hot melt adhesive, tea tree leaf oil, hydro-colloid, Calendula officinalis flower extract, and a moisture absorbent. The hydrocolloid layer may be formed on the base material film 141 by adhesive coating.

When the adhesive layer 142 is formed as a hydrocolloid layer, it can improve skin moisturizing and skin adhesion, and the functional material 143 located on an upper surface of the hydrocolloid layer which comes in contact with the skin can supply moisture and nutrients to the skin, which results in skin wrinkle improvement, whitening effect, melasma and freckles improvement effect, medical effects such as wound treatment, etc.

The functional material 143 may include 85 to 97% by weight of hyaluronic acid and 3 to 15% by weight of a skin improving material. When the amount of hyaluronic acid is less than the above range, the moisturizing effect and skin beauty effect due to hyaluronic acid may be insignificant, and when the amount of hyaluronic acid exceeds 97% by weight, the effect of improving skin whitening, melasma, and freckles due to the skin improving material may be insignificant. The skin improving material may supply moisture and nutrients to the skin together with hyaluronic acid to give a skin beauty effect, and specifically, the skin improving material includes one or more of hydroquinone, kojic acid, albutin, placenta, tretinoin, AHA (alpha-hydroxy acid), azelaic acid, licorice extract, and Centella asiatica extract, but may include additional materials in addition to the above examples. In addition, the functional material 143 may include a medical liquid material having a therapeutic effect.

Hereinafter, the functional patch manufacturing apparatus 1000 according to one embodiment of the present disclosure will be described with reference to FIGS. 2 to 5. The functional patch manufacturing apparatus 1000 according to one embodiment of the present disclosure includes a dispensing section 1100, a drying section 1200, and a recovery section 1300.

The dispensing section 1100 is a section for applying the functional material 143 to the base patch 140. The dispensing section 1100 includes a main roller 1110, a separation roller 1120, a cover film collection roller 1130, a dispenser 1140, a functional material supply device P, and a controller C.

The main roller 1110 is configured to unwind and release the multi-layer patch film 100. The multi-layer patch film 100 is wound on the main roller 1110. Then, when the main roller 1110 operates and rotates, the multi-layer patch film 100 wound thereon is unwound, and one end of the multi-layer patch film 100 is released.

The separation roller 1120 is configured to separate the cover film 130 from the multi-layer patch film 100. When the multi-layer patch film 100 released from the main roller 1110 reaches the separation roller 1120, the cover film 130 is separated from the multi-layer patch film 100 by rotation of the separation roller 1120.

The cover film collection roller 1130 is configured to collect the cover film 130 separated by the separation roller 1120. After the cover film 130 is separated by the separation roller 1120, the separated cover film 130 is wound on the cover film collection roller 1130 while the cover film collection roller 1130 rotates to be collected.

The dispenser 1140 is a device for applying the functional material 143 to the base patch 140. The dispenser 1140 may include a nozzle 1141 for discharging the functional material 143. The dispenser 1140 applies the functional material 143 to the base patch 140 of the multi-layer patch film 100 from which the cover film 130 is separated by the separation roller 1120. Specifically, the functional material 143 is applied to the adhesive layer 142 of the base patch 140 exposed to the outside through the guide hole 121 in the multi-layer patch film 100 released from the separation roller 1120. The nozzle 1141 applies the functional material 143 to the adhesive layer 142 when the guide hole 121 is located at a position corresponding thereto. The nozzle 1141 is disposed above the multi-layer patch film 100, and the functional material 143 may be applied to the guide hole 121 from above.

In addition, for more accurate application of the functional material 143, the dispenser 1140 or the nozzle 1141 may be driven in an up-down direction. More specifically, when the guide hole 121 is located at a position corresponding to the dispenser 1140 or the nozzle 1141, the functional material 143 may be applied when the dispenser 1140 or the nozzle 1141 is driven downward to be closer to the guide hole 121. In this case, the functional material 143 can be applied in a more accurate position and shape.

The dispenser 1140 may include a plurality of nozzles 1141. As described above, n number of guide holes 121 may be formed in a row in the guide film 120 along the width direction of the base film 110, and the plurality of nozzles 1141 may be arranged so that n number of nozzles 1141 form a row to correspond to the n number of guide holes 121. In this case, the functional material 143 may be applied to then number of guide holes 121 at the same time. The n number of nozzles 1141 may be disposed in a state fixed to each other in the dispenser 1140. For example, one space (not shown) in which the functional material 143 is accommodated is formed inside the dispenser 1140, and the n number of nozzles 1141 may be configured to communicate with the one space (not shown).

Meanwhile, when the dispenser 1140 includes n number of nozzles 1141 and the dispenser 1140 or the nozzles 1141 are driven in the up-down direction as described above, the n number of nozzles 1141 can be driven in the up-down direction at the same time.

The functional material supply device P is a device for supplying the functional material 143 to the dispenser 1140. The functional material supply device P may includes a container for storing the functional material 143, a tube connecting the container and the dispenser 1140, and a pump for supplying the functional material 143 from the container to the dispenser 1140.

The controller C is a device for controlling the application operation of the dispenser 1140 and the supply operation of the functional material supply device P. The controller C may be disposed on the side of the dispensing section 1100.

The drying section 1200 is a device for drying the functional material 143 in the base patch 140 with the functional material 143 coated thereon discharged from the dispensing section 1100. The drying section 1200 supplies heat to the functional material 143 to dry the functional material 143. The drying section 1200 includes a heat supply unit 1220 for supplying heat. The drying section 1200 includes a transfer unit 1210. The transfer unit 1210 is configured to transfer the multi-layer patch film 100 including the base patch 140 from which the cover film 130 has been removed and to which the functional material 143 has been applied. Accordingly, the multi-layer patch film 100 is moved by the transfer unit 1210, and the functional material 143 is dried by heat through the heat supply unit 1220 during the transfer process. The base patch 140 in which the applied functional material 143 has been dried is referred to as a finished patch.

The heat supply unit 1220 of the drying section 1200 according to one embodiment of the present disclosure may be a hot air supply unit 1230. The hot air supply unit 1230 is configured to have a device (not shown) for generating hot air outside, blow the hot air into the drying section 1200 by a blower (not shown), and supply the hot air to the functional material 143. The functional material 143 can be dried by the hot air.

In FIG. 2, it is shown that three drying sections 1200 as shown in FIG. 4 are provided, but, as needed, one drying section 1200 may be provided, and two or more drying sections 1200 may be provided.

The recovery section 1300 is a device for recovering the multi-layer patch film 100 discharged from the drying section 1200. That is, the recovery section 1300 is a device for recovering the multi-layer patch film 100 on which the finished patch is disposed in a state in which the cover film 130 is removed. The recovery section 1300 may include a guide roller 1320 and a recovery roller 1310. While rotating, the recovery roller 1310 may wind and recover the multi-layer patch film 100 on which the finished patch is disposed. The guide roller 1320 is a roller that guides the movement of the multi-layer patch film 100 when the multi-layer patch film 100 is moved to the recovery roller 1310.

Meanwhile, the separation roller 1120, the transfer unit 1210 of the drying section 1200, and the guide roller 1320 of the recovery section 1300 may all be positioned at the same height. In this case, while moving from the dispensing section 1100, through the drying section 1200, to the recovery section 1300, the multi-layer patch film 100 can be moved stably without being bent or deformed.

Hereinafter, with reference to FIG. 6, the operation of the dispenser 1140 of the functional patch manufacturing apparatus 1000 according to one embodiment of the present disclosure will be described in detail.

The dispenser 1140 and the functional material supply device P may be controlled by the controller C to apply the functional material 143 at predetermined time intervals. For example, the multi-layer patch film 100 moves at a specific speed V, and when the guide holes 121 are arranged along the longitudinal direction of the multi-layer patch film 100 at an interval of a first length L1, a time interval at which the adjacent guide holes 121 pass through the nozzle 1141 is determined as a first time T1. In this case, the speed V may be the magnitude of a linear speed of the main roller 1110 or a linear speed of the separation roller 1120. Further, the controller C may control the dispenser 1140 so that the functional material 143 is applied from the nozzle 1141 at the interval of the first time T1.

In addition, as described above, in the guide film 120, the guide holes 121 are arranged so that n number of guide holes form a row and m number of guide holes form a column, and a plurality of guide films 120 may be disposed on the base film 110. In this case, in the guide films 120 adjacent to each other, when the shortest distance between the guide hole 121 of one guide film 120 and the guide hole 121 of the other guide film 120 is referred to as a second length L2, a time for the multi-layer patch film 100 to move by the second length L2 is determined as a second time T2. In this case, n number of nozzles 1141 may be disposed in the dispenser 1140, and in the nozzle 1141, the functional material 143 is repeatedly applied m times at the interval of the first time T1, and then the functional material 143 may be applied again after the second time T2. And, this process may be repeated. That is, the controller C may control the dispenser 1140 to sequentially repeat an application step in which the dispenser 1140 repeatedly applies through the nozzle 1141 the functional material 143 m times at the interval of the first time T1, and a stopping step in which the application of the dispenser 1140 is stopped during the second time T2. In this case, the functional material 143 can be accurately applied to each guide hole 121 even with a simple configuration and setting.

Meanwhile, perforated holes (not shown) may be formed in a region of the base film 110 outside the guide film 120 based on the width direction of the multi-layer patch film 100. The perforated holes (not shown) may be symmetrically formed on both sides of the guide film 120 based on the width direction of the multi-layer patch film 100. In addition, the perforated holes (not shown) may be formed outside the guide film 120 based on the longitudinal direction of the multi-layer patch film 100, or formed central side of the guide films 120.

The dispenser 1140 may include a perforated hole detection sensor (not shown) for sensing the perforated hole (not shown). The perforated hole detection sensor (not shown) may be disposed at a position corresponding to the perforated hole (not shown). The perforated hole detection sensor (not shown) may sense the perforated hole (not shown) through transmittance of light or sound waves.

Whether or not to start an initial operation of the dispenser 1140 may be determined by the perforated hole (not shown) and the perforated hole detection sensor (not shown). Specifically, a distance between a specific perforated hole (not shown) and the guide hole 121 disposed closest thereto rearward based on the moving direction of the multi-layer patch film 100 is measured in advance, and based on the distance, a time taken when the multi-layer patch film 100 is moved by the distance is calculated. Initially, the dispenser 1140 is in a state where the application operation is stopped. Then, when a perforated hole (not shown) is sensed by the perforated hole detection sensor (not shown), the dispenser 1140 starts to apply the functional material 143 after the above time. In this case, the nozzle 1141 of the dispenser 1140 can more accurately apply the functional material 143 to the guide hole 121.

In addition, a plurality of perforated holes (not shown) may be formed at regular intervals along the longitudinal direction of the multi-layer patch film 100. In this case, while the multi-layer patch film 100 passes through the dispenser 1140, the position of the guide film 120 or the guide hole 121 can be accurately recognized at regular intervals, based on which the application operation of the dispenser 1140 can be corrected, so that the nozzle 1141 of the dispenser 1140 can more accurately apply the functional material 143 to the guide hole 121.

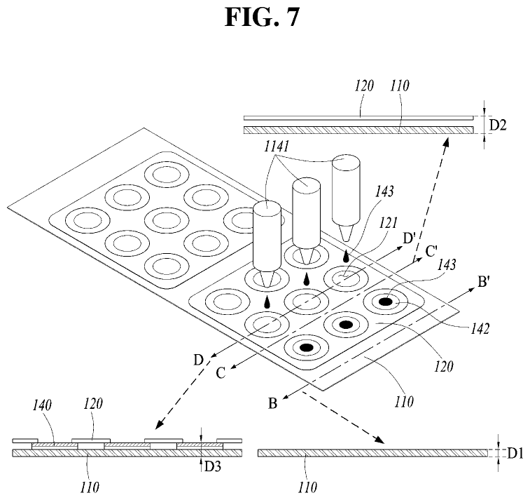

FIG. 7 is a perspective view illustrating the operation of a dispensing section according to another embodiment of the present disclosure.

Hereinafter, referring to FIG. 7, a functional patch manufacturing apparatus 1000 according to another embodiment of the present disclosure will be described in detail. The functional patch manufacturing apparatus 1000 according to another embodiment of the present disclosure further includes a thickness measurement sensor, and the dispenser 1140 operates according to the measurement result of the thickness measurement sensor. Hereinafter, redundant descriptions of the same parts as those of the functional patch manufacturing apparatus 1000 according to one embodiment of the present disclosure will be omitted.

The thickness measurement sensor is a sensor for measuring a thickness of each part of the multi-layer patch film 100 in real time. The thickness measuring sensor may be disposed between the separation roller 1120 and the dispenser 1140 or at a position corresponding to the dispenser 1140. The thickness measurement sensor is preferably located just before the dispenser 1140 between the separation roller 1120 and the dispenser 1140 in consideration of the little time required for the control process of the controller C.

The multi-layer patch film 100 has a different thickness for each part. In the case of a portion where only the base film 110 exists, the thickness is a first thickness D1 equal to a thickness of the base film 110, and a portion where the guide film 120, not the guide hole 121, is located, has a second thickness D2 greater than the first thickness D1, and a portion where the guide hole 121 is formed has a third thickness D3 greater than the first thickness D1 and smaller than the second thickness D2. The thickness measurement sensor can separately measure the first thickness D1, the second thickness D2, and the third thickness D3, and which part of the multi-layer patch film 100 is passing through the dispenser 1140 can be determined based on the measurement results.

When it is measured by the thickness measurement sensor that the portion of the second thickness D2 passes through the dispenser 1140, the controller C determines that the portion of the guide hole 121 passes through the dispenser 1140, and controls the dispenser 1140 to apply the functional material 143. In this case, the dispenser 1140 can accurately apply the functional material 143 to the guide holes 121 even if the distance between the guide holes 121 or the distance between the guide films 120 varies.

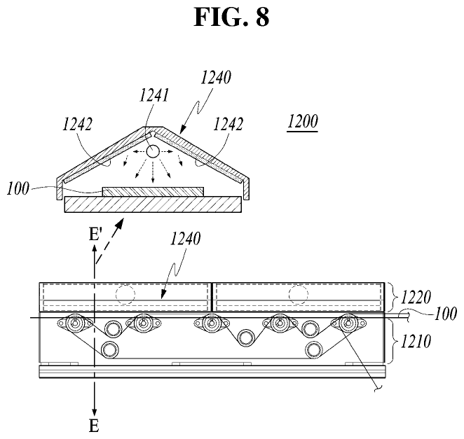

FIG. 8 shows a drying section including a near-infrared heater according to still another embodiment of the present disclosure.

Hereinafter, with reference to FIG. 8, a functional patch manufacturing apparatus 1000 according to still another embodiment of the present disclosure will be described in detail. In the functional patch manufacturing apparatus 1000 according to still another embodiment of the present disclosure, the drying section 1200 includes a near-infrared heater 1240. Hereinafter, redundant descriptions of the same parts as those of the functional patch manufacturing apparatus 1000 according to one embodiment of the present disclosure will be omitted.

The near-infrared heater 1240 may include a near-infrared lamp 1241 and a reflector 1242. The near-infrared lamp 1241 emits near-infrared rays. The emitted near-infrared rays may transfer heat to the functional material 143 through a radiation phenomenon. The reflector 1242 may reflect the emitted near-infrared rays so that the near-infrared rays are evenly transmitted to the functional material. In this case, the reflector 1242 may be disposed on both sides of the near-infrared lamp 1241, which allows the near-infrared rays to be supplied to the functional material 143 very uniformly.

The functional material 143 may be warmed up to a specific temperature due to the near-infrared rays and radiation caused by the near-infrared rays. The specific temperature may be set to less than 50° C., preferably to 40° C. or more and 45° C. or less. In addition, the functional material 143 can be dried evenly as a whole within a short period of time due to the transmission of light. In this case, the functional material 143 can be dried very efficiently and with high quality while preventing deformation of the functional material 143.

In addition, when the heat supply unit 1220 of the drying section 1200 includes the near-infrared heater 1240, unlike the hot air supply unit 1230 of the drying section 1200 according to one embodiment of the present disclosure, the configuration and structure of the heat supply unit 1220 can be simplified and thermal efficiency thereof can be increased while minimizing power consumption.

The near-infrared ray lamp 1241 of the near-infrared heater 1240 may be a microbeam lamp (not shown) that emits light in a microbeam region. The light in the microbeam region means light having a wavelength of 0.8 μm to 4 μm, and the near-infrared ray lamp 1241 of the present disclosure may preferably emit light of a wavelength of 0.8 μm to 2 μm among the light in the above microbeam region. Such a microbeam lamp (not shown) takes less than 1 second to reach the maximum output and can be heated very quickly, minimizing production and process time, and having a thermal efficiency of 85% to 90%, which is very economical.

Although one embodiment of the present disclosure has been described above, those skilled in the art may variously modify and change the present disclosure by adding, changing, or deleting the components within the scope of the present disclosure without departing from the idea of the present disclosure described in the claims, and it will be said that such modifications and changes are also included within the scope of the present disclosure.

### DESCRIPTION OF REFERENCE NUMERALS

100: multi-layer patch film
110: base film
120: guide film
121: guide hole
130: cover film
140: base patch
141: base material film
142: adhesive layer
143: functional material
1000: functional patch manufacturing apparatus
1100: dispensing section
1110: main roller
1120: separation roller
1130: cover film collection roller
1140: dispenser
1141: nozzle
1200: drying section
1210: transfer unit
1220: heat supply unit
1230: hot air supply unit
1240: near-infrared heater
1241: near-infrared lamp
1242: reflector
1300: recovery section
1310: recovery roller
1320: guide roller
P: functional material supply device
C: controller

What is claimed is:

1. A functional patch manufacturing apparatus which includes a dispensing section, a drying section, and a recovery section and manufactures a functional patch by processing a plurality of base patches disposed in a multi-layer patch film, wherein:
the multi-layer patch film includes:
a base film having a width and extending in a longitudinal direction;
a guide film disposed on the base film, the guide film having at least one guide hole smaller than the base patch, having a width less than or equal to that of the base film, and having a length shorter than that of the base film; and
a cover film formed and disposed to cover the base film and the guide film,
wherein the base patch is attached to the guide film and the base film at a position corresponding to the guide hole between the guide film and the base film, so that at least a part of the base patch is exposed to the outside of the guide film,
wherein the base film, the base patch, the guide film and the cover film are sequentially stacked,
wherein the dispensing section includes:
a main roller on which the multi-layer patch film is initially wound, the wound multi-layer patch film being unwound from the main roller during operation;
a separation roller for separating the cover film from the multi-layer patch film unwound from the main roller;
a cover film collection roller for collecting the cover film separated from the separation roller;
a dispenser for applying a functional material to a portion of the base patch exposed through the guide hole in the multi-layer patch film separated by the separation roller from above;
a functional material supply device for supplying the functional material to the dispenser;
a controller for controlling the application operation of the dispenser and the supply operation of the functional material supply device,
wherein the drying section includes a heat supply unit for supplying heat to the functional material applied to the base patch,
wherein the base patch is converted into a finished patch in which the functional material is dried by the heat supplied from the heat supply unit to be discharged, and
wherein the recovery section includes a recovery roller for recovering the multi-layer patch film in which the finished patch is disposed in a state in which the cover film is removed.

2. The functional patch manufacturing apparatus of claim 1, wherein the base patch includes a base material film and an adhesive layer bonded to the base material film, and the functional material is applied to the adhesive layer.

3. The functional patch manufacturing apparatus of claim 2, wherein the base material film is a thermoplastic polyurethane resin material, and the adhesive layer is a hydrocolloid layer.

4. The functional patch manufacturing apparatus of claim 1, wherein the functional material contains 85 to 97% by weight of hyaluronic acid.

5. The functional patch manufacturing apparatus of claim 1, wherein in the guide film, n number of guide holes are formed in a row in a width direction of the base film, and
wherein the dispenser includes n number of nozzles disposed to correspond to the guide holes of the row, and the n number of nozzles simultaneously apply the functional material.

6. The functional patch manufacturing apparatus of claim 1, wherein in the guide film, n number of guide holes are formed in a row in a width direction of the base film, and m number of guide holes are formed in a column in a longitudinal direction of the base film.

7. The functional patch manufacturing apparatus of claim 1, wherein the controller controls the dispenser to sequentially repeat an application step in which the dispenser applies the functional material m times at a first time interval and a stopping step in which the application of the dispenser is stopped for a second time after the application step.

8. The functional patch manufacturing apparatus of claim 6, further comprising:
a thickness sensor for measuring the thickness of each part of the multi-layer patch film in real time,
wherein the controller analyzes a thickness value measured by the thickness sensor, and controls the dispenser so that the dispenser applies the functional material when it is determined that the guide hole is at a position corresponding to the dispenser.

9. The functional patch manufacturing apparatus of claim 1, wherein the heat supply unit is a hot air supply unit for blowing hot air to the functional material applied to the base patch.

10. The functional patch manufacturing apparatus of claim 1, wherein the heat supply unit is a near-infrared heater for supplying near-infrared radiant heat to the functional material applied to the base patch.
